(19) United States
(12) Patent Application Publication
(10) Pub. No.: US 2024/0351954 A1
(43) Pub. Date: Oct. 24, 2024

(54) WATER TOLERANT ENZYMATIC STRUCTURAL MATERIAL

(71) Applicant: Worcester Polytechnic Institute, Worcester, MA (US)

(72) Inventors: Nima Rahbar, Weston, MA (US); Shuai Wang, Worcester, MA (US); Suzanne Scarlata, Worcester, MA (US)

(21) Appl. No.: 18/639,498

(22) Filed: Apr. 18, 2024

Related U.S. Application Data
(60) Provisional application No. 63/460,437, filed on Apr. 19, 2023.

(51) Int. Cl. C04B 35/52; B28B 11/24; C04B 35/626; C04B 35/64; C12P 3/00

(52) U.S. Cl. CPC C04B 35/52; C04B 35/6264; C04B 35/62655; C04B 35/64; C12P 3/00; B28B 11/24; C04B 2235/422; C04B 2235/442; C04B 2235/48; C04B 2235/5436; C04B 2235/6027; C04B 2235/606; C04B 2235/656

(57) ABSTRACT

A carbon-negative Engineering Structural Material (ESM) has a compressive strength approaching that of concrete and relies on a carbon-absorbing enzyme for crystalline formations formed cooperatively with a porous structure to achieve load-bearing properties. A tough scaffold forms through capillary suspension, a technique that utilizes capillary forces to concentrate particles in a liquid matrix. Carbonic anhydrase, a zinc-containing enzyme extracted from bovine erythrocytes, is harnessed to grow mineral materials, and the capillary suspension is used to create a construction material, including sand and a polymer. This combination enables the incorporation of precipitated calcium minerals into the structure, resulting in the development of water-resistant and load-bearing construction materials.

### RELATED APPLICATIONS

This patent application claims the benefit under 35 U.S.C. § 119(e) of the U.S. Provisional Patent App. No. 63/460,437, filed Apr. 19, 2023, entitled “WATER TOLERANT ENZYMATIC CONSTRUCTION MATERIAL,” incorporated herein by reference in its entirety.

### STATEMENT OF FEDERALLY SPONSORED RESEARCH AND DEVELOPMENT

This invention was made with government support under grant No. NSF 2223664, awarded by the National Science Foundation. The government has certain rights in the invention.

### BACKGROUND

Concrete is the most consumed construction material in the world and is employed in widespread infrastructure, such as bridges, buildings, and roadways, because of its outstanding durability, mechanical strength, versatility, and cost-efficiency. The World Economic Forum reported that buildings account for 40% of the global energy consumption and 33% of greenhouse gas emissions. Carbon emissions related to the use of Portland cement (the critical functional ingredient in concrete) in the concrete industry pose significant challenges to low-carbon development strategies, which has driven researchers to look for alternatives to Portland cement.

### SUMMARY

A carbon-negative Engineering Structural Material (ESM) has a compressive strength that approaches that of concrete. It relies on a carbon-absorbing enzyme for crystalline formation formed cooperatively with a porous structure to achieve load-bearing properties. A tough scaffold is formed through capillary suspension, a technique that utilizes capillary forces to concentrate particles in a liquid matrix. Carbonic anhydrase, a zinc-containing enzyme extracted from bovine erythrocytes, is harnessed to grow mineral materials, and a capillary suspension is used to create a construction material, including sand and a polymer. This combination enables the incorporation of precipitated calcium minerals into the structure, resulting in the development of a water-resistant and load-bearing carbon-negative structural material.

The configurations herein are based, in part, on the observation that more carbon-favorable substitutes for concrete are sought by increasingly stringent building practices. Bacterial and microbial approaches for concrete substitutes are attractive. However, conventional approaches to concrete substitutes suffer from shortcomings of water vulnerability, leading to their inability to survive without a layer of water-resistant protection. Accordingly, these configurations substantially overcome the prior art problems of moisture and combine water durability with high compressive strength for a carbon-negative material. The disclosed ESM shows higher water durability than other biological-inspired construction materials with a substantial mechanical strength of 25 MPa-28 MPa, which is close to the minimum compressive strength of structural concrete made with hydraulic cement, making it a promising candidate for construction applications. ESM production consumes a net carbon dioxide of approximately 18.5 lb. per cubic yard, lending it a carbon-negative property, in sharp contrast to conventional Portland cement-based cementitious materials.

In further detail, the method for forming a structural material includes adding oil to a granular solid and forming a crystalline mixture immiscible with the oil. This is followed by combining the crystalline mixture with oil, and the granular solid forms a capillary suspension, providing a scaffold for crystals from the crystalline mixture, which may then be formed into a mold. The capillary suspension is heated until the hydrochar forms a water-tolerant structural material in a shape prescribed by the mold.

### BRIEF DESCRIPTION OF THE DRAWINGS

The foregoing and other objects, features, and advantages of the invention will be apparent from the following description of particular embodiments of the invention, as illustrated in the accompanying drawings, in which reference characters refer to the same parts throughout the different views. The drawings are not necessarily to scale, instead emphasizing being placed upon illustrating the principles of the invention.

FIG. 1 shows a context diagram of the carbon-negative formation and life cycle of the Engineering Structural Material (ESM), as disclosed herein;

FIG. 2 shows the process flow for ESM formation, as shown in FIG. 1;

FIGS. 3A and 3B are images of the ESM showing the porous structure in cooperation with crystals of calcium;

FIG. 4 shows a molecular diagram of the curing process into hydrochar; and

FIGS. 5A and 5B show the performance testing results for the compressive strength.

### DETAILED DESCRIPTION

Carbon-neutral and carbon-negative approaches to structural materials are among the frontier scientific issues. Previous efforts to generate construction materials that sequester rather than generate CO₂ during production have been proposed in the copending U.S. patent application Ser. No. 17/943,548, filed Sep. 13, 2022, entitled “ENZYMATIC CONSTRUCTION MATERIAL” (ECM) incorporated herein by reference in its entirety. The disclosed sand-based material contains carbonic anhydrase (CA), a metalloenzyme that catalyzes the interconversion of carbon dioxide (CO₂) and bicarbonate (HCO₃⁻) ions that accelerates the equilibrium toward mineral growth on a polymer scaffold to provide strength to carry a load. A subsequent development incorporating nanoparticles to achieve photothermal curing ability and serve as a multifunctional thermal element is disclosed in the U.S. application Ser. No. 18/110,484, filed Feb. 16, 2023, entitled “ENZYMATIC CONSTRUCTION MATERIAL FOR REPAIR AND CORROSION RESISTANCE AND DURABILITY,” (ECM-n) also incorporated by reference. The compressive strength of ECM and ECM-n was approximately 12 MPa, and their cracks under fracture loading could self-heal under ambient conditions or laser induction. However, similar to other biologically inspired structural materials, ECM may encounter issues with moisture and humidity. In general, conventional biological structural materials tend to use hydrophilic polymers as scaffolds to incubate bacteria and proteins, which exposes the weakness of their mechanical properties to water/high humidity.

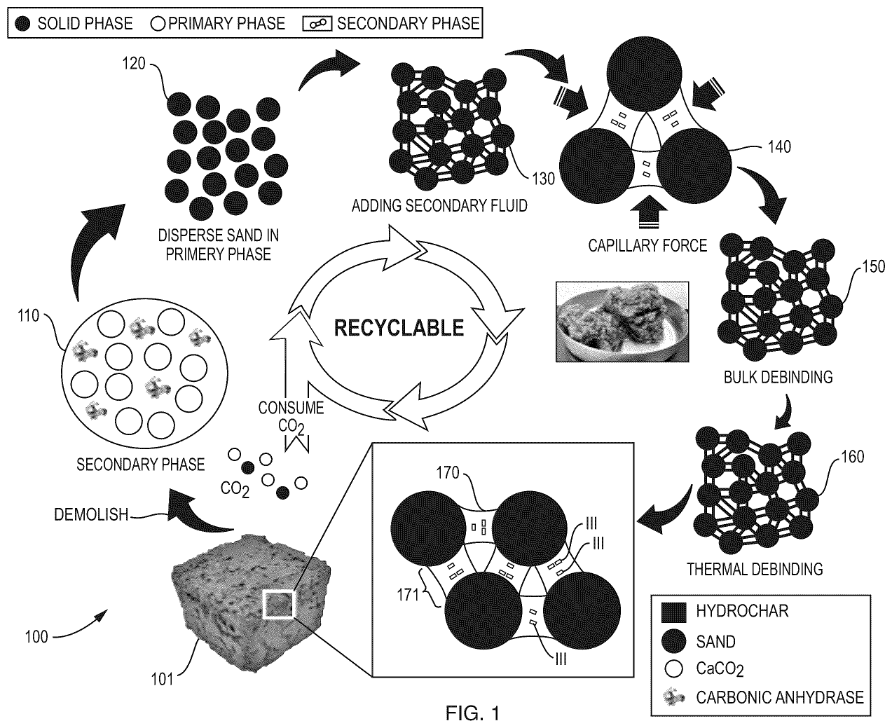

FIG. 1 shows a context diagram of the carbon-negative formation and life cycle of the Engineering Structural Material (ESM), as disclosed herein. Referring to FIG. 1, Process 100 and the lifecycle formation of a building material with a hydrophobic structure formulation are shown. Process 100 reveals an ESM that harnesses the principles of capillary suspension to fabricate a carbon backbone microstructure by bonding sand particles at optimal porosity to stabilize calcium carbonate minerals. FIG. 1 shows a schematic of the source materials, component distribution, thermal curing, and details of the fabrication mechanism. This results in a water-resistant, scalable, and low-carbon structural material with a high compressive strength ESM compound 101. ESM is a ternary particle-liquid-liquid system composed of particles dispersed in an immiscible liquid that can form various structures depending on the ratio of the three components and their material properties. A mineral (such as calcium) is first precipitated using an enzymatic method and then incorporated into a secondary fluid to form secondary phase 110, as discussed further below. The formulation of the ESM compound is guided by the concentration of the secondary phase within the suspension, which can adjust the designated structural porosity, eventually resulting in ternary composites with predesigned mechanical properties. Based on the American Concrete Institute (ACI) standard, the minimum compressive strength requirement for structural concrete is set at 17 MPa, which is lower than both the raw compressive strength and the water-immersed treatment compressive strength of the disclosed ESM. The physical and chemical components change during the thermal curing of the material, forming bonds that provide strength. An interfacial transition zone (ITZ) formed between the scaffold and aggregate within the materials can be observed using focused ion-beam scanning electron microscopy (FIB-SEM).

Expressed as a cyclic evolution, the solid ESM compound includes a solid or primary phase 120 formed from granular solids, such as sand and oil. Sand is analogous to the aggregate in conventional concrete. A calcium source, including carbon dioxide, is formed by enzymatic reactions. CO₂ forms calcium carbonate (CaO₃) crystals, which combine with secondary fluid 130. The addition of the secondary phase forms an immiscible dispersion of the solid phase in capillary suspension 140 with the primary phase and calcium source. The material rapidly forms a slurry 150 with a semi-solid consistency, which can be formed into molds, similar to the fabrication techniques of precast concrete structures. The application of heat cures capillary suspension 160 to form a water-resistant structural material, or ESM 101. The heating or curing process forms hydrochar bonds 170 from calcium carbonate 111 (from the secondary phase 110), filling the porous structure 171 resulting from the capillary suspension.

ESM 101 exhibits outstanding water durability, compressive strength, buildability, and low carbon content, which are superior to those of any current biological structural materials, and its carbon emissions are significantly lower than those of Portland cement concrete. The properties of ESM are promising for potential applications in precast shapes such as bricks, roof shingles, and more substantial molded shapes currently fulfilled by precast methods, positioning it as a viable substitute for conventional concrete.

It has been estimated that an average of 927 kg (2044 lb.) of CO₂ is released every 1000 kg (2205 lb.) Portland cement produced in the United States. The typical CO₂ emissions associated with concrete production amount to approximately 500 pounds per cubic yard.

Life cycle assessment (LCA) considers all life cycle phases of materials, including the production of raw materials, construction and service phases, demolition and dismantling, and the disposal or recycling of waste materials. Using the manufacturer's data to estimate the LCA of carbon sequestration from concrete carbonation, researchers have considered the most significant part of the entire life cycle of concrete materials, including raw materials, cement production, aggregate production, and transport. Contemporary experimental evidence demonstrates ESM as a potential substitute for concrete.

Carbonation of the ESM 101 is the process in which atmospheric carbon dioxide (CO₂) reacts with calcium ions through the enzyme catalyst. The catalyzed reaction is the first step in ESM production, and the precipitated calcium carbonate results in the sequestration of CO₂. Bovine carbonic anhydrase (CA) may be employed as a metalloenzyme, in which a zinc ion located at the active center of the protein structure facilitates the dissociation of hydrogen ions from water, which then binds to carbon dioxide to produce carbonate (Eq. 1 below). This process commences immediately and is completed within 6 hours under ambient conditions. The percentage of calcium carbonate in ESM (mass) was calculated using Eq. 2:

$${Ca}_{( aq )}^{2 +} + {CO}_{2} + {H}_{2} {O}_{( 1 )} \to {CaCO}_{3 ( s )} \qquad ( 1 )$$

$${W}_{CaCO 3} \% = \frac{{W}_{CaCO 3}}{{W}_{CaCO 3} + {W}_{hydrochar} + {W}_{aggregate}} \times 100 \% \qquad ( 2 )$$

The environmental benefits are substantial. One cubic yard of ESM production consumes 18.5 lb. of CO₂, whereas concrete releases 498.0 lb. CO₂ per cubic yard. The transfer efficiency, indicating the degree of oxidation of the hydrochar from sucrose within 3.5 hours at the thermal curing base, was determined from the experimental results. The transfer efficiency is given by Eq. 3. The total carbon content remained constant throughout the thermal curing process, and residual carbon was oxidized to carbon dioxide.

$${C}_{c} = ( \frac{{C}_{hydrochar}}{{C}_{sucrose}} ) \times 100 \% \qquad ( 3 )$$

The system boundary of LCA includes stages from raw material extraction to the end-of-life stage (demolition). The service stage and demolition were ignored because full carbonation was assumed to be achieved during the lifespan of the production stage. The carbon footprint calculation of ESM simplifies carbonation. It is assumed that most of the stages were the same as those of concrete and did not consider additional benefits from ESM: the high open porosity of ESM provides a large number of nucleation sites for calcium carbonate crystal growth, which potentially assists in additional carbon sequestration during service; furthermore, calcium carbonate, hydrochar, and aggregates exhibit inert properties, making them resistant to decomposition at the end of their lifespan. The van der Waals bond between the hydrochar and sand in ESM is significantly more straightforward to break down. Hence, the material can be readily recycled compared to traditional concrete with covalent bonding. The density of ESM is 1.82 g/cm³ compared to concrete, 2.4 g/cm³, which mitigates energy use during transportation.

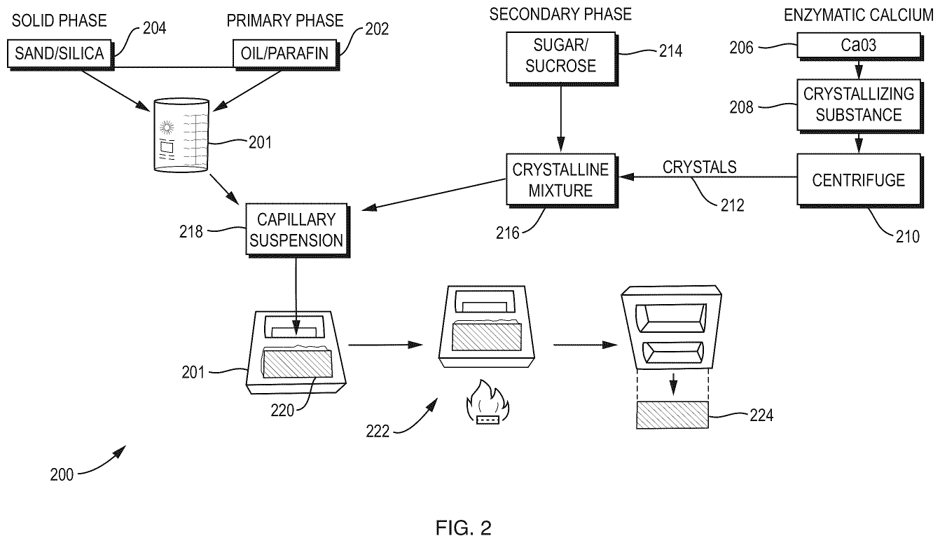

FIG. 2 shows a process flow of 200 for the formation of ESM, as shown in FIG. 1, depicting the role of calcium yield through CA and the role of hydrochar formed in the patterned suspension. Referring to FIGS. 1 and 2, the method for forming the ESM 101 as a construction material includes, at step 201, adding an oil 202 (primary phase) to a granular solid 204 (solid phase). The primary (bulk) phase of the capillary suspension includes a low-viscosity paraffin oil, specifically Carolina paraffin oil, with a boiling point ranging from 326 to 407° C. and a specific gravity within the range of 0.83-0.860. To form fresh sand surfaces and obtain results closer to actual engineering conditions, all-purpose sand aggregates were sourced to obtain a washed, properly graded granular solid that meets the American Society for Testing and Materials (ASTM) C33 specifications. Subsequently, the sand was sieved to obtain particles with dimensions of 75 μm and 150 μm, which were deemed suitable for experimental purposes. In a particular configuration, the granular solids have a particle size in a range between fine and coarse sand of 75 μm and 150 μm with a weight ratio of 1.3:1. The sand aggregate was washed with a 1% acetic acid aqueous solution for three cycles, followed by five cycles of DI water cleaning, until the pH of the waste exceeded 6.5. The density of the sand particles was determined to be 1.22 g/cm³.

A calcium source is combined with carbonic anhydrase (CA) to generate enzymatic reactions, as depicted in step 206. This includes the formation of a crystallizing substance by adding an enzyme, such as CA, to a calcium solution to form calcium carbonate crystals, as depicted in step 208. The calcium solution is then agitated by centrifugation or a similar approach at step 210 to settle and extract calcium carbonate crystals 212. The enzyme added to the solution to react with carbon dioxide forms the crystals and provides favorable carbon chemistry.

In the example configuration, the secondary phase is sourced from sucrose, preferably reagent-grade quality, and was prepared as a 50% sucrose aqueous solution (6 g of sucrose was gently stirred into 6 g of water for five minutes sucrose aqueous solution), as disclosed at step 214. The density of the aqueous sucrose solution is 1.31 g/cm³. A crystalline mixture is formed in step 216 by combining the calcium carbonate crystals 212 with the aqueous sucrose 214 to define the secondary phase solution. In the example configuration, to prepare the secondary phase, 3.3 g of the calcium carbonate crystals (2.93 g/mL at 25° C., was introduced into the 12 g 50% sucrose solution, while stirring continued for an additional period to ensure thorough homogenization. The density of the secondary phase is approximately 1.46 g/cm³.

The secondary phase is selected based on its immiscibility with the primary phase and the formation of a scaffold for crystals from the capillary suspension. The sucrose solution in step 214 forms a crystalline mixture immiscible with the oil. The crystalline mixture is combined with the oil and the granular solid (sand) to form a capillary suspension, providing a scaffold for crystals from the crystalline mixture, as depicted in step 218. The immiscible combination generates a slurry from the capillary suspension of the immiscible oil and aqueous sucrose solution.

In the example configuration, the capillary suspensions were prepared using a systematic approach with a dispersion of 10 vol % silica sand within 85 vol % paraffin oil; however, other immiscible oils or substances may suffice. A thorough mixing step was executed using a stirring plate operating at 500 rpm for one minute, before introducing 5 vol % of the secondary phase. To obtain homogeneity, a supplementary manual mixing step was performed 5 min prior to material casting. Notably, the resultant material exhibited a ternary system (solid-liquid-liquid phase), classifying it as being in the pendular state.

The formed slurry has a semi-solid consistency, and step 220 forms the slurry into mold 201 with a predetermined shape. The mold is heated until the capillary suspension forms hydrochar, thereby forming a water-tolerant construction material, as depicted in step 222. Mold 201 is heated for at least an hour at a temperature between 190° C.-250° C. to cure the slurry in the mold. A particular configuration involves heating the mold for 50-70 minutes at a temperature between 90° C.-110°, followed by heating at a temperature between 190° C.-250° for 80-100 minutes. Alternate heating approaches may be employed; however, it is preferable to heat the capillary suspension to induce hydrothermal carbonization without producing biochar. Following heating, the molded ESM formed from the cured slurry was removed from the mold, as depicted in step 224. Through the curing process, the scaffold receives crystals formed from the calcium source and the enzymatic reactions to form the ESM 101. The final result is the formation of a high-compressive-strength structural material produced from a carbon-negative process.

As shown above, the microstructural transformation of the ESM 101 is driven by a capillary suspension. As the thermal curing progressed, the residual primary phase underwent gasification and was released from the system. Simultaneously, the secondary liquid underwent hydrothermal carbonization (HTC) in an oven chamber, reacting with air to produce hydrochar. This process increases the solid volume of the system, forming solid bridges that interconnect the spaces between the solid phases, resulting in the formation of a consolidated mass.

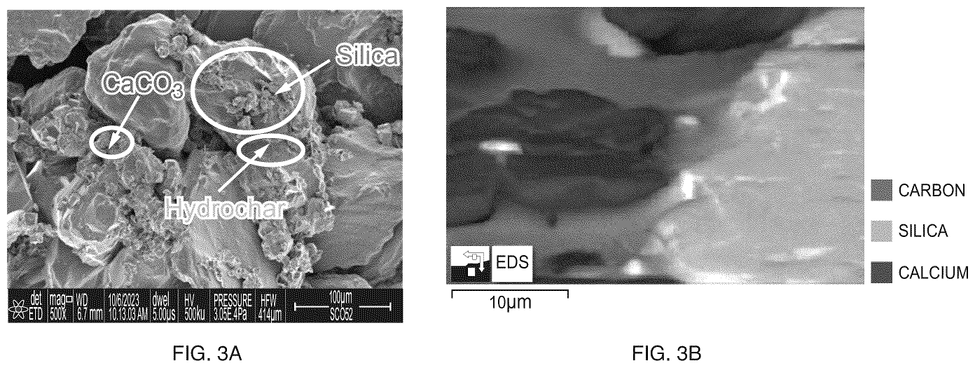

FIGS. 3A and 3B are images of the ESM showing the porous structure in cooperation with crystals of calcium. Referring to FIGS. 1-3B, within the cured ESM, calcite (calcium carbonate) crystals served as part of the bridges in the hydrochar, connecting with sand aggregates, as shown in FIGS. 3A and 3B. The deposition of secondary phases in the microstructure is distinct, with calcite dispersed in the micropores of the hydrochar, creating bridges with the hydrochar around the sand aggregate. SEM analysis in FIG. 3A reveals that the scaffold bridges predominantly consist of calcite crystals incorporated within the hydrochar to link the silica sand. In ESM, the composite of calcite and hydrochar bridges is a crucial factor in providing compressive strength. Within a specific range, increasing the dosage of calcite crystals or hydrochar enhances compressive strength,

Referring again to step 281, the formation of the slurry is defined by a green body suspension of ESM prepared using the solid, secondary, and primary phases by thorough mixing, which is a significant feature in designing a high-property capillary suspension material. From the perspective of molecular mechanics, the rheology of a green body is usually controlled by the interparticle and liquid-solid interfaces between the attractive van der Waals forces and repulsive electrostatic forces. Macroscopically, the surface tension at the liquid-air interface and liquid-solid interfaces influences the stability of the liquid bridge between particles. The viscosity of the liquid phase increases from resistance to flow within the liquid of the suspension, and the shape and size of the solid particles form different flow behaviors, which are crucial factors that affect the overall rheology. Brownian motion can influence the distribution and movement of the particles in the liquid phase. The random thermal motion of particles can lead to collisions and interactions that affect the stability, arrangement, and flow properties of the capillary structures.

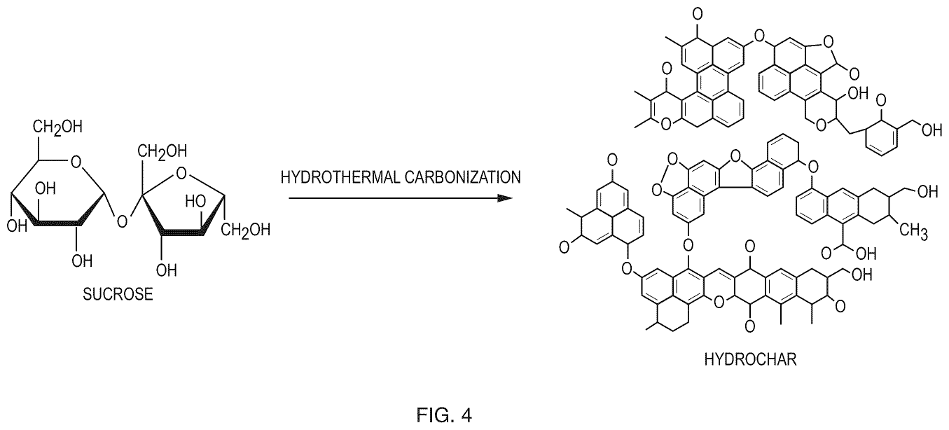

FIG. 4 shows a molecular diagram of the curing process of sucrose into the hydrochar. Hydrothermal carbonization is a process in which organic compounds are heated in the presence of water at elevated temperatures. In the disclosed example for producing ESM 101, hydrothermal carbonization is primarily driven by sucrose hydrolysis, which involves dehydration and polymerization. This process converts the sucrose structure into a more carbon-rich and stable material known as hydrochar, as shown in FIG. 4. It should be noted that based on the configurations herein, a small amount of paraffin oil (0.25 g) remained in the 1 cubic inch ESM (approximately 33 g). In FIG. 7b, the TGA (Thermogravimetric analysis) diagram verifies that there was no significant weight loss at temperatures below 100° C. (92.5% remaining), and a substantial weight loss occurred between 170° C. and 200° C. Above 200° C., the rate of weight loss tended to stabilize, indicating that the degree of curing and chemical activity of ESM were mild.

The hydrochar tends to exhibit a wide range of physicochemical and morphological variations. It is generally assumed that the primary chemical form of the hydrochar is C₃H₂O. As the C/H ratio changes during thermal curing, the stoichiometric ratios in the decarbonation reaction were affected. This alteration has implications for the coefficients of carbon, hydrogen, and oxygen in the reaction as well as for the overall estimated carbon threshold results, as described in Equation 4. Adherence to a predetermined curing temperature and duration is recommended for engineering applications.

$${C}_{12} {H}_{22} {O}_{11 ( s )} + {O}_{2} + {H}_{2} {O}_{( 1 )} \to {C}_{3} {H}_{2} {O}_{( s )} + {CO}_{2} + {H}_{2} O \qquad ( 4 )$$

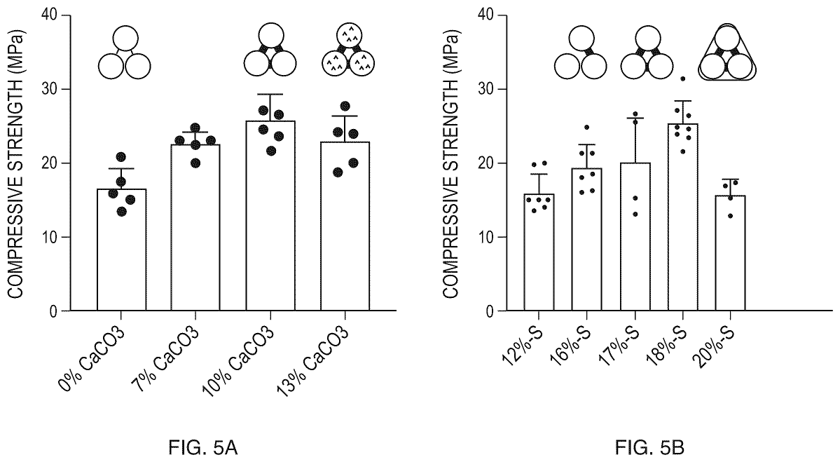

FIGS. 5A and 5B show performance testing results for compressive strength. Referring to FIGS. 1 and 5A-5B, the compressive strengths of the ESM under different conditions are shown. FIG. 5A shows the compressive strength of ESM with different densities of the secondary phases. The term of “X % CaCO3” represents the ESM containing X % CaCO3 by weight. FIG. 5B shows the compressive strength of ESM with different dosages of the secondary phase. The term of “X %-S” represents the ESM containing X % aqueous sucrose by weight with an equal amount of CaCO3 particles.

The balance between the volumes of the coarse and fine aggregates, along with the weight ratio of the secondary phase to the solid phase, plays an important role in formulating materials with superior strength, impressive buildability, and homogeneity. A well-designed slurry in a capillary suspension results in a stable structure that prevents settling in the mold, ensuring a uniform slurry density from top to bottom. The processing route (FIG. 1) involves the formation of capillary suspensions using a combination of enzymatically precipitated calcium carbonate and sucrose aqueous solution as the secondary fluid, mixed with the primary and solid phases. After thermal curing, the resulting capillary bridges contain calcium carbonate crystals and oxidized sucrose product-hydrochar, which were present between the solid phases (silica sand particles). As part of the secondary phase, calcium carbonate crystals contribute to the addition of capillary suspension and strength, enhance the effect of agglomeration, and serve as high-strength components in the final product.

The disclosed capillary suspension technique can be employed with any type of secondary fluid and primary phase, ensuring a contrast in their density differences and immiscibility. A well-designed ESM is instrumental in attaining diverse engineering objectives encompassing different types and shapes of aggregates, diverse ratios between phases, and varying compositions within the phases. The ESM preparation process is straightforward and can be efficiently scaled up for mass production. It should be apparent that future ESM iterations would be well suited for use in load-bearing and non-load-bearing structures, where it can offset CO₂ emissions. Because of its lower density compared to concrete, transported prepatterned ESM bricks can minimize labor and cost. Despite these advantages, several challenges remain unaddressed. Improving the interface between the aggregate and scaffold would enhance both compressive and tensile strengths. Finding a sustainable replacement for paraffin oil could significantly reduce the CO₂ emissions. Additionally, refining the hydrochar structure to reduce the porosity may enhance the compressive strength, making it more applicable to reinforced rebar structures.

While the system and methods defined herein have been particularly shown and described with references to embodiments thereof, it will be understood by those skilled in art that various changes in form and details may be made therein without departing from the scope of the invention encompassed by the appended claims.

What is claimed is:

1. A method for forming a structural material, comprising:
adding an oil to a granular solid;
forming a crystalline mixture immiscible with the oil;
combining the crystalline mixture with the oil and the granular solid to form a capillary suspension providing a scaffold for crystals from the crystalline mixture; and
heating of the capillary suspension until the formation of hydrochar, thereby forming a water-tolerant construction material.

2. The method of claim 1 further comprising forming the crystalline mixture by:
adding an enzyme to a solution for reacting with carbon dioxide for forming crystals; and
combining the crystals with a sugar solution to form the crystalline mixture.

3. The method of claim 1 further comprising forming the crystalline mixture by:
adding an enzyme to a calcium solution to form calcium carbonate crystals;
agitating the calcium solution for settling and extracting the calcium carbonate crystals; and
combining the calcium carbonate crystals with an aqueous sucrose solution to form the crystalline mixture.

4. The method of claim 1 further comprising forming a scaffold for crystals in the crystalline mixture from the capillary suspension formed from the oil, which defines the structural porosity for receiving the crystals.

5. The method of claim 3 further comprising:
generating a slurry from the capillary suspension of the immiscible oil and aqueous sucrose solution; and
forming the slurry into a mold with a predetermined shape.

6. The method of claim 1, wherein the water-tolerant structural material defines a carbon-negative process.

7. The method of claim 2 wherein the enzyme is carbonic anhydrase (CA).

8. The method of claim 1 wherein the sugar solution is an aqueous sucrose solution.

9. The method of claim 5 further comprising:
heating the mold for at least an hour at a temperature between 190° C.-250° C. for curing the slurry in the mold; and
removing the cured slurry from the mold.

10. The method of claim 5 further comprising heating the mold for 50-70 minutes at temperatures between 90° C.-110°, followed by heating at temperature between 190° C.-250° for 80-100 minutes.

11. The method of claim 1 further comprising heating the capillary suspension to induce hydrothermal carbonization without producing biochar.

12. A solid engineering structural material comprising
a solid phase formed from granular solids,
a calcium source formed from enzymatic reactions including carbon dioxide,
a primary phase including an oil, and
a secondary phase forming an immiscible dispersion of the solid phase in a capillary suspension with the primary phase and the calcium source,
the capillary suspension cured via heating to form the water-resistant structural material.

13. The material of claim 12, wherein the capillary suspension is heated to induce hydrothermal carbonization of the crystals maintained in the capillary suspension.

14. The material of claim 12, wherein the secondary phase is selected based on its immiscibility with the primary phase and a formation of a scaffold for crystals from the capillary suspension.

15. The material of claim 14, wherein the scaffold for crystals receives crystals formed from the calcium source and the enzymatic reactions.

16. The material of claim 12, wherein the calcium source is combined with carbonic anhydrase (CA) to generate the enzymatic reactions.

17. The material of claim 11 wherein the granular solids have a particle size in a range between of fine and coarse sand of 75 μm and 150 μm with a weight ratio of 1.3:1.

18. A method for forming a carbon-negative, high-compressive-strength structural material comprising:
combining an enzyme with a calcium solution to form calcium carbonate crystals;
separating the calcium carbonate crystals to form a crystalline mixture;
adding the crystalline mixture to an aqueous sugar solution;
adding an oil to a solid phase defined by granular silica;
combining the aqueous sugar solution to the granular silica and oil to form a capillary suspension in a slurry form from the, oil and aqueous sugar solution, the oil immiscible with the aqueous sugar solution;
forming the slurry into a mold, the mold defining a shape of the high compressive strength structural material;
heating the mold including the slurry for at least one hour at a temperature of at least 95° C. to stabilize the formed slurry, and for at least another hour at a temperature of at least 190° C. for the formation of hydrochar, thereby curing the slurry in the defined shape.
and
releasing the high-compressive-strength structural material from the mold.

19. The method of claim 18, wherein the aqueous sugar solution includes sucrose, and the oil is paraffin oil.

20. The method of claim 18, wherein the formation of the high-compressive-strength structural material involves a carbon-negative process.
